SEQUENCE LISTING (5 sequences)

SEQ ID NO: 1 - DNA, 1038 bp, Lactobacillus fermentum
ctacacatgcaagtcgaacgcgttggcccaattgattgatggtgcttgcacctgattgattttggtcgccaacgagtggcggacgggtgagtaacacgtaggtaacctgcccagaagcgggggacaacatttggaaacagatgctaataccgcataacaacgttgttcgcatgaacaacgcttaaaagatggcttctcgctatcacttctggatggacctgcggtgcattagcttgttggtggggtaatggcctaccaaggcgatgatgcatagccgagttgagagactgatcggccacaatgggactgagacacggcccatactcctacgggaggcagcagtagggaatcttccacaatgggcgcaagcctgatggagcaacaccgcgtgagtgaagaagggtttcggctcgtaaagctctgttgttaaagaagaacacgtatgagagtaactgttcatacgttgacggtatttaaccagaaagtcacggctaactacgtgccagcagccgcggtaatacgtaggtggcaagcgttatccggatttattgggcgtaaagagagtgcaggcggttttctaagtctgatgtgaaagccttcggcttaaccggagaagtgcatcggaaactggataacttgagtgcagaagagggtagtggaactccatgtgtagcggtggaatgcgtagatatatggaagaacaccagtggcgaaggcggctacctggtctgcaactgacgctgagactcgaaagcatgggtagcgaacaggattagataccctggtagtccatgccgtaaacgatgagtgctaggtgttggagggtttccgcccttcagtgccggagctaacgcattaagcactccgcctggggagtacgaccgcaaggttgaaactcaaaggaattgacgggggcccgcacaagcggtggagcatgtggtttaattcgaagctacgcgaagaaccttaccaggtcttgacatcttgcgccaaccctagagatagggcgtttccttcgggaacgcaatgacaggtggt

SEQ ID NO: 2 - DNA, 900 bp, Lactobacillus fermentum
tgcaagtcgaacgcgttggcccaattgattgatggtgcttgcacctgattgattttggtcgccaacgagtggcggacgggtgagtaacacgtaggtaacctgcccagaagcgggggacaacatttggaaacagatgctaataccgcataacagcgttgttcgcatgaacaacgcttaaaagatggcttctcgctatcacttctggatggacctgcggtgcattagcttgttggtggggtaacggcctaccaaggcgatgatgcatagccgagttgagagactgatcggccacaatgggactgagacacggcccatactcctacgggaggcagcagtagggaatcttccacaatgggcgcaagcctgatggagcaacaccgcgtgagtgaagaagggtttcggctcgtaaagctctgttgttaaagaagaacacgtatgagagtaactgttcatacgttgacggtatttaaccagaaagtcacggctaactacgtgccagcagccgcggtaatacgtaggtggcaagcgttatccggatttattgggcgtaaagagagtgcaggcggttttctaagtctgatgtgaaagccttcggcttaaccggagaagtgcatcggaaactggataacttgagtgcagaagagggtagtggaactccatgtgtagcggtggaatgcgtagatatatggaagaacaccagtggcgaaggcggctacctggtctgcaactgacgctgagactcgaaagcatgggtagcgaacaggattagataccctggtagtccatgccgtaaacgatgagtgctaggtgttggagggtttccgcccttcagtgccggagctaacgcattaagcactccgcctgggggagtacgaccgcaaggttgaaactcaaaggaat

SEQ ID NO: 3 - DNA, 1050 bp, Lactobacillus fermentum
ggcccaattgattgatggtgcttgcacctgattgattttggtcgccaacgagtggcggacgggtgagtaacacgtaggtaacctgcccagaagcgggggacaacatttggaaacagatgctaataccgcataacagcgttgttcgcatgaacaacgcttaaaagatggcttctcgctatcacttctggatggacctgcggtgcattagcttgttggtggggtaacggcctaccaaggcgatgatgcatagccgagttgagagactgatcggccacaatgggactgagacacggcccatatcctacgggaggcagcagtagggaatcttccacaatgggcgcaagcctgatggagcaacaccgcgtgagtgaagaagggtttcggctcgtaaagctctgttgttaaagaagaacacgtatgagagtaactgttcatacgttgacggtatttaaccagaaagtcacggctaactacgtgccagcagccgcggtaatacgtaggtggcaagcgttatccggatttattgggcgtaaagagagtgcaggcggttttctaagtctgatgtgaaagccttcggcttaaccggagaagtgcatcggaaactggataacttgagtgcagaagagggtagtggaactccatgtgtagcggtggaatgcgtagatatatggaagaacaccagtggcgaaggcggctacctggtctgcaactgacgctgagactcgaaagcatgggtagcgaacaggattagataccctggtagtccatgccgtaaacgatgagtgctaggtgttgggagggtttccgcccttcagtgccggagctaacgcattaagcactccgcctggggagtacgaccgcaaggttgaaactcaaaggaattgacgggggcccgcacaagcggtggagcatgtggtttaattcgaagctacgcgaagaccttaccaggtcttgacatctgcgccaaccctagagatagggcgtttccttcgggaacgcaatgacaggtggtgcatggtcgtcgtcagctcgtgtcgtgagaatgtgggt

SEQ ID NO: 4 - DNA, 924 bp, Lactobacillus fermentum
ggtgcttgcacctgattgattttggtcgccaacgagtggcggacgggtgagtaacacgtaggtaacctgcccagaagcgggggacaacatttggaaacagatgctaataccgcataacagcgttgttcgcatgaacaacgcttaaaagatggcttctcgctatcacttctggatggacctgcggtgcattagcttgttggtggggtaacggcctaccaaggcgatgatgcatagccgagttgagagactgatcggccacaatgggactgagacacggcccatactcctacgggaggcagcagtagggaatcttccacaatgggcgcaagcctgatggagcaacaccgcgtgagtgaagaagggtttcggctcgtaaagctctgttgttaaagaagaacacgtatgagagtaactgttcatacgttgacggtatttaaccagaaagtcacggctaactacgtgccagcagccgcggtaatacgtaggtggcaagcgttatccggatttattgggcgtaaagagagtgcaggcggttttctaagtctgatgtgaaagccttcggcttaaccggagaagtgcatcggaaactggataacttgagtgcagaagagggtagtggaactccatgtgtagcggtggaatgcgtagatatatggaagaacaccagtggcgaaggcggctacctggtctgcaactgacgctgagactcgaaagcatgggtagcgaacaggattagataccctggtagtccatgccgtaaacgatgagtgctaggtgttggagggtttccgcccttcagtgccggagctaacgcattaagcactccgcctggggagtacgaccgcaaggttgaaactcaaaggaattgacgggggcccgcacaagcggtggagcatgtggtttaattcgaagctacgcgaagaa

SEQ ID NO: 5 - DNA, 911 bp, Lactobacillus fermentum
tgcaagtcgaacgcgttggcccaattgattgatggtgcttgcacctgattgattttggtcgccaacgagtggcggacgggtgagtaacacgtaggtaacctgcccagaagcgggggacaacatttggaaacagatgctaataccgcataacaacgttgttcgcatgaacaacgcttaaaagatggcttctcgctatcacttctggatggacctgcggtgcattagcttgttggtggggtaatggcctaccaaggcgatgatgcatagccgagttgagagactgatcggccacaatgggactgagacacggcccatactcctacgggaggcagcagtagggaatcttccacaatgggcgcaagcctgatggagcaacaccgcgtgagtgaagaagggtttcggctcgtaaagctctgttgttaaagaagaacacgtatgagagtaactgttcatacgttgacggtatttaaccagaaagtcacggctaactacgtgccagcagccgcggtaatacgtaggtggcaagcgttatccggatttattgggcgtaaagagagtgcaggcggttttctaagtctgatgtgaaagccttcggcttaaccggagaagtgcatcggaaactggataacttgagtgcagaagagggtagtggaactccatgtgtagcggtggaatgcgtagatatatggaagaacaccagtggcgaaggcggctacctggtctgcaactgacgctgagactcgaaagcatgggtagcgaacaggattagataccctggtagtccatgccgtaaacgatgagtgctaggtgttggagggtttccgcccttcagtgccggagctaacgcattaagcactccgcctggggagtacgaccgcaaggttgaaactcaaaggaattgacgggggccc

(19) United States
(12) Patent Application Publication
(10) Pub. No.: US 2016/0348191 A1
(43) Pub. Date: Dec. 1, 2016

(54) LACTIC ACID BACTERIUM LACTOBACILLUS FERMENTUM ISOLATED FROM ADULTS IN LONGEVITY VILLAGE, HELPFUL FOR DEFECATION

(71) Applicant: PL BIO CO., LTD., Seoul (KR)

(72) Inventors: Yeonhee LEE, Seoul (KR); Jong-su PARK, Daejeon (KR); Kyungsoo PAEK, Seoul (KR); Ki-Hyun AHN, Seongnam-si (KR); Enju SHIN, Seoul (KR); Hyunjin HONG, Seoul (KR); Hak Mi LEE, Seoul (KR); Minyoung LEE, Seoul (KR); Hyun-Jung SHIN, Daejeon (KR); Young-Hoon CHO, Daejeon (KR)

(21) Appl. No.: 14/906,892

(22) PCT Filed: Jul. 21, 2014
(86) PCT No.: PCT/KR2014/006611

§ 371 Date: Jan. 21, 2016

(30) Foreign Application Priority Data
Jul. 22, 2013 (KR) 10-2013-0086153

(51) Int. Cl. C12R 1/225; A23L 33/135; A61K 35/747

(52) U.S. Cl. CPC C12R 1/225; A61K 35/747; A23L 33/135; A61K 2035/115

(57) ABSTRACT

The invention relates to a novel strain, Lactobacillus fermentum strain, and a use thereof. Specifically, novel Lactobacillus fermentum PL9988, novel Lactobacillus fermentum PL9037, novel Lactobacillus fermentum PL9038, novel Lactobacillus fermentum PL9039 and novel Lactobacillus fermentum PL9040 strains, isolated from adults in a longevity village, and culture fluids thereof: have no antibiotic resistance transfer; have excellent intestinal cell adhesiveness, acid resistance and bile resistance; inhibit harmful pathogenic organisms in intestines; and have an immunity-enhancing effect, an endotoxin shock suppression effect and an antioxidant effect. In particular, Lactobacillus fermentum PL9988 strain significantly satisfies all of the above-mentioned conditions as a probiotic, and thus can be effectively used as a probiotic for intestinal health and defecation improvement and enhancement, or as a health food.

### TECHNICAL FIELD

The present invention relates to a novel Lactobacillus fermentum strain isolated from adults in a longevity village which is able to enhance and promote defecation, and a use of probiotic of the strain.

### BACKGROUND ART

From old times, it is known that the relationship between foods which people eat and health is important. In particular, it is revealed that intestinal microorganisms may change depending on the foods which people eat. According to a result of the research showing that the intestinal microorganisms have a lot to do with people's health and diseases, thus determining the level of immunity (Round JL. Mazmanian SK, 2009, The gut microbiota shapes intestinal immune responses during health and disease. Nature Reviews Immunology 9, 313-323) and obesity (Turnbaugh PJ. et. al., 2006. The obesity-associated gut microbiome with increased capacity for energy harvest. Nature 444, 1027-1031), it is known that an intestinal microorganism of each person closely affect the body condition of each person.

Which kinds of lactic acid bacterium each person have may be determined by the lactic acid bacterium which is taken into a body for the first few months after the person is born. Thereafter, since the lactic acid bacteria in the body is not continuously maintained in the intestine. intestinal bacteria may be determined by the lactic acid bacteria which are taken from foods. For example, Lactobacillus acidophilus which is frequently found in western people who consume a lot of milk and mild product is not found in oriental people. In contrast, Lactobacillus plantarum which is frequently found in oriental people has been rarely found in western people. The above two lactic acid bacteria are found very often in the dairy products in western countries and the fermented foods in oriental countries, respectively.

Most of representative lactic acid bacteria on the market in Korea are lactic acid bacteria which were originally isolated from western people or their foods, which are rarely found in the intestines of Korean people. Therefore typical Korean lactic acid bacteria may be developed from adults who live in a famous longevity belt in Korea and have healthy intestinal activities (regular bowel movements). Various functions as a probiotic of this lactic acid bacterium may be characterized. In particular, a novel functional lactic acid bacterium which may be helpful to healthy intestinal movement without any antibiotic resistance transfer which is a worldwide issue should be developed and used as an effective probiotic.

The probiotic was defined as a singular or combined strains of a microorganism which is alive and has an effect on hosts by improving the characteristics of intestinal microorganisms when an appropriate amount of the probiotic is taken. In recent years, it is generally defined as a microorganism formulation containing foods and feeds or a dietary additive, (Salminen S J, Gueimonde M, Isolauri E. 2005. Probiotics that modify disease risk. J Nutr. 135: 1294-1298) to enhance the health of a human body or an animal.

The probiotic-lactic acid bacterium may be categorized into Lactobacillus, Lactococcus, Streptocossus, Leuconostoc, Pediococcus, and Bifidobacterium and is known to play important roles in microorganism infection prevention in intestines and urogenital organs, intestinal health maintenance, alleviation of constipation, inhibition of harmful bacterium proliferation, anti-cancer effect, immunity enhancement, decrease in cholesterol, production of CLA (Conjugated Linoleic Acid), and inhibition of Helicobacter pylori, etc., which means that it play an important role in enhancing a human body health.

Good probiotics must have followings characteristics: safety; a tolerance to acid and bile; an adhesive ability to intestinal epithelium; an inhibition on pathogenic bacteria; immune enhancement; and only intrinsic antibiotic resistance which cannot be transferred.

Bacteria in general may have antibiotic resistances in two ways. First, it is an intrinsic resistance which is not transferred, namely, it is a viviparous resistance. For example, many lactic acid bacteria may be vancomycin resistant due to the specific structure and thickness of cell wall. This intrinsic resistance is not transferred, and an intrinsic gene is not transferred to other bacteria. Second, it is an extrinsic resistance. This extrinsic resistance is referred to a resistance which is obtained from a resistance gene combined with plasmid or transposon and can enter other bacteria. In this case, the gene of such a resistance bacterium may easily transfer resistance to other bacteria.

It is previously believed that the lactic acid bacterium with strong resistance is a good one. So the antibiotic resistant lactic acid bacteria which survived after treatment with various antibiotics have been considered good. However, many researchers have reported that resistance genes of lactic acid bacteria to various antibiotics may be transferred to intestinal bacteria, thus causing a problem with resistance. To prevent this problem, western countries have banned the lactic acid bacteria with resistance problem from commercialization, if the resistance lactic acid bacteria are able to transfer gene. In case of Korea, an article on the resistance transfer of antibiotics was recently added to a food material determination criteria of microorganism by Ministry of Food and Drug Safety, Korea. The article has been used as a food material determination criteria of microorganism.

Survey on intestinal microorganism distributions in residents who live in health longevity farm villages or cities and who are over 40 years was performed. The result showed there is a difference between the residents living at the cities and at the longevity farm villages with respect to the distributions of beneficial bacteria, for example, Lactobacillus, Lactococcus, etc. which are helpful to health was 0.56%: 1.355% with respect to all the intestinal bacteria, and in case of Lactococcus, it was 0.02%: 0.1%, which meant that as compared to the residents living at the cities, the residents living at the longevity village was 3 to 5 times higher. No lactic acid bacteria are not known yet, which may satisfy all the important characteristics such as non-transferable antibiotic resistance, resistance to acid and bile which are necessary for probiotic, an adhesion to an intestine epithelium, an inhibition activation with respect to pathogenic bacteria, an anti-oxidation activation, and an immunity enhancement effect.

The inventors of the present invention have conducted a research in an effort to find out a safe lactic acid bacterium which does not have any antibiotic resistance transfer possibility. As a result of the above research, a novel lactobacillus fermentum strain was isolated from healthy adults who live in longevity villages in Korea. The above strain and its culture medium thereof had good intestinal cell adhesiveness, an acid resistance and a bile resistance, and there was not any external resistance with respect to antibiotic. There were an intestinal harmful pathogenic bacterium inhibition effect, an immunity enhancement effect, an anti-toxicity shock inhibition effect, and an anti-oxidation effect. In particular, since the above novel Lactobacillus fermentum PL9988 strain meaningfully satisfied all the above conditions as probiotics, it was confirmed that the novel Lactobacillus fermentum PL9988 strain or its culture medium could be used as a probiotic or a health food so as to enhance and promote defecation, thus completing the present invention.

### SUMMARY OF THE INVENTION

### Disclosure of Invention

Accordingly, it is an object of the present invention to provide a novel Lactobacillus fermentum strain which is isolated from adults who are living in a longevity belt region and have a regular intestinal movement (regular bowel movements).

It is another object of the present invention to provide a probiotic composition and a health food for a defecation enhancement and promotion from the active ingredients of novel lactobacillus fermentum strain or its culture medium.

### Technical Solution

To achieve the above objects, there is provided a novel Lactobacillus fermentum strain.

To achieve the above objects, there is provided a probiotic composition, which may include any of a novel Lactobacillus fermentum PL9988 deposited as a deposit number of KCTC12624BP; a novel Lactobacillus fermentum PL9037; a novel Lactobacillus fermentum PL9038; a novel Lactobacillus fermentum PL9039; a novel Lactobacillus fermentum PL9040; and a culture medium thereof.

To achieve the above objects, there is provided a health food, which may include any of a novel Lactobacillus fermentum PL9988 deposited as a deposit number of KCTC12624BP; a novel Lactobacillus fermentum PL9037; a novel Lactobacillus fermentum PL9038; a novel Lactobacillus fermentum PL9039; a novel Lactobacillus fermentum PL9040; and a culture medium thereof.

To achieve the above objects, there is provided a use of any of a novel Lactobacillus fermentum PL9988 deposited as a deposit number of KCTC12624BP; a novel Lactobacillus fermentum PL9037; a novel Lactobacillus fermentum PL9038; a novel Lactobacillus fermentum PL9039; a novel Lactobacillus fermentum PL9040; and a culture medium thereof, which are contained for the use as a probiotic composition.

To achieve the above objects, there is provided a use of any of a novel Lactobacillus fermentum PL9988 deposited as a deposit number of KCTC12624BP; a novel Lactobacillus fermentum PL9037; a novel Lactobacillus fermentum PL9038; a novel Lactobacillus fermentum PL9039; a novel Lactobacillus fermentum PL9040; and a culture medium thereof, which are contained for the use as a health food.

### Advantageous Effects

The novel Lactobacillus fermentum strain PL9988, novel Lactobacillus fermentum strain PL9037, novel Lactobacillus fermentum strain PL9038, novel Lactobacillus fermentum strain PL9039, novel Lactobacillus fermentum strain PL9040 and the culture medium thereof according to the present invention have a good intestine cell adhesiveness and a good acid resistance and a good bile resistance and don't have any danger in terms of an antibiotic resistance transfer and are able to inhibit harmful pathogenic bacteria in intestines and have an effect on an immunity enhancement, an anti-toxicity short inhibition, and an anti-oxidation. In particular, as the novel Lactobacillus fermentum strain PL9988 strain can meaningfully satisfy all the above conditions as probiotic, the novel Lactobacillus fermentum strain PL9988 strain may be usefully used as a probiotic or a health food which is able to enhance defecation and intestinal health state.

### BRIEF DESCRIPTION OF DRAWINGS

FIG. 1 is a view illustrating a relationship between the 16S rRNA base sequences of a Lactobacillus fermentum PL9988 strain and a standard strain CECT562 (Lactobacillus fermentum strain CECT562) of a Lactobacillus fermentum.

FIG. 2 is a view illustrating a relationship between the 16S rRNA base sequences of a Lactobacillus fermentum PL9037 strain and a standard strain CECT562 of a Lactobacillus fermentum.

FIG. 3 is a view illustrating a relationship between the 16S rRNA base sequences of a Lactobacillus fermentum PL9038 strain and a standard strain CECT562 of a Lactobacillus fermentum.

FIG. 4 is a view illustrating a relationship between the 16S rRNA base sequences of a Lactobacillus fermentum PL9039 strain and a standard strain CECT562 of a Lactobacillus fermentum.

FIG. 5 is a view illustrating a relationship between the 16S rRNA base sequences of a Lactobacillus fermentum PL9040 strain and a standard strain CECT562 of a Lactobacillus fermentum.

FIG. 6 is a view illustrating an observed pattern of a Lactobacillus fermentum PL9988.

FIG. 7 is a view illustrating an inhibited zone size with respect to harmful bacteria of a Lactobacillus fermentum PL9988 strain.

FIG. 8 is a view illustrating a Lactobacillus fermentum PL9988 strain attached to intestinal cells.

FIG. 9 is a view illustrating a confirmed effect on an immunity enhancement of a Lactobacillus fermentum PL9988 strain with respect to immunity cells processed with a lipopolysaccharide (LPS).

FIG. 10 is a view illustrating an anti-oxidation capability of a Lactobacillus fermentum PL9988 strain.

### MODES FOR CARRYING OUT THE INVENTION

The present invention will be described in detail below.

The present invention provides a novel Lactobacillus fermentum strain.

The strain according to the present invention is any of a novel Lactobacillus fermentum strain PL9988 which has a 16S rRNA base sequence marked as a sequence number 1 and is deposited with a deposit number of KCTC12624BP, a novel Lactobacillus fermentum strain PL9037 which has a 16S rRNA base sequence marked as a sequence number 2, a novel Lactobacillus fermentum strain PL9038 which has a 16S rRNA base sequence marked as a sequence number 3, a novel Lactobacillus fermentum strain PL9039 which has a 16S rRNA base sequence marked as a sequence number 4 and a novel Lactobacillus fermentum strain PL9040 which has a 16S rRNA base sequence marked as a sequence number 5.

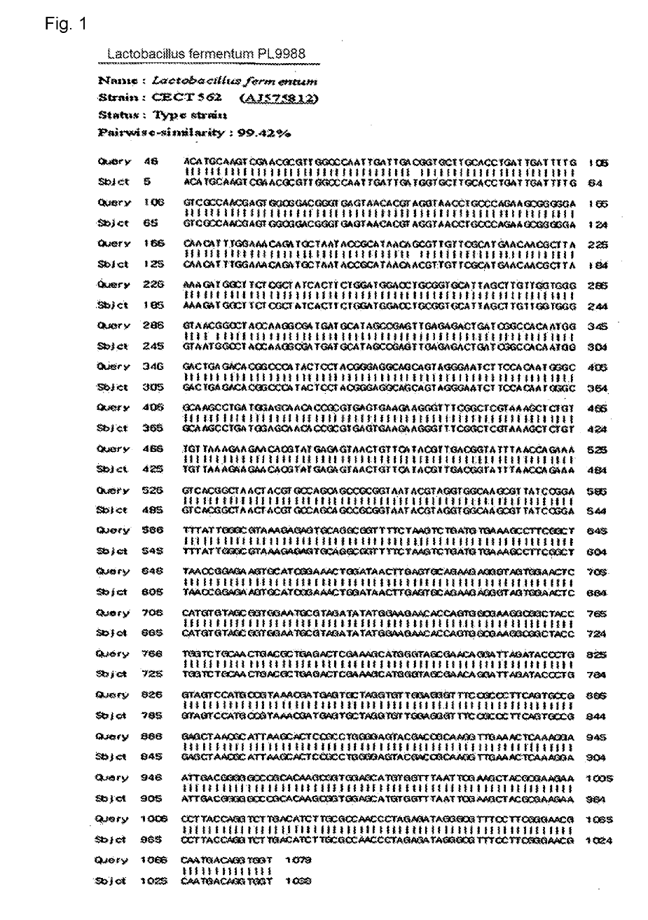

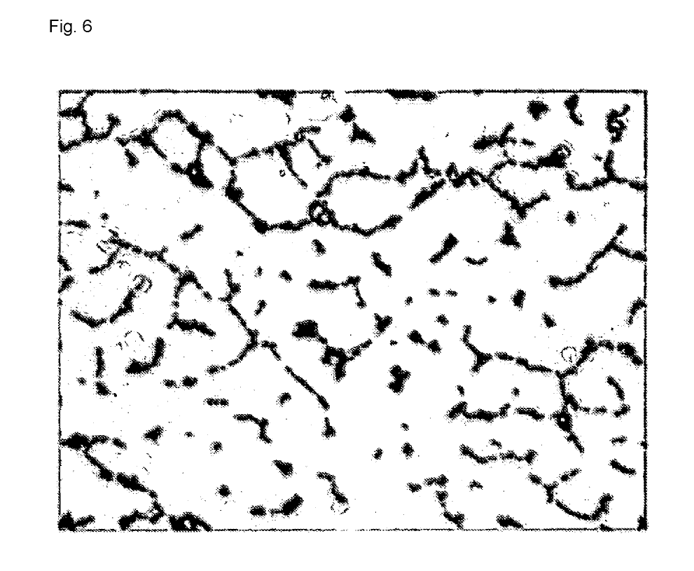

According to a preferred exemplary embodiment of the present invention, the strain was isolated from the sample of human feces from healthy adults who were living in a longevity belt and have regular defecation and identified, and the strain was confirmed as a novel strain which showed a 99.42% homology with the standard strain CECT562 (Lactobacillus fermentum strain CECT562) of the Lactobacillus fermentum through the 16S rRNA base sequence analysis, and it was confirmed that the above strain had a typical Lactobacillus fermentum-shaped Gram positive bacillus (refer to FIGS. 1 and 6). The strain in the novel Lactobacillus fermentum was named a Lactobacillus fermentum PL9988 and was deposited at the microorganism resource center of Korea Research Institute of Bioscience and Biotechnology on Jul. 16, 2014 (Deposit number KCTC12624BP).

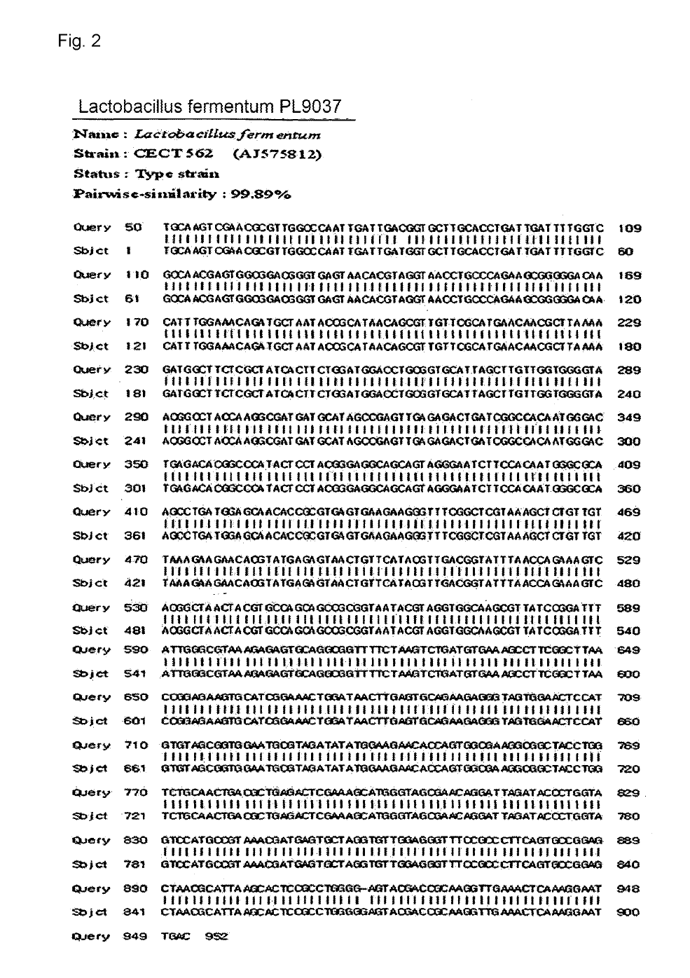

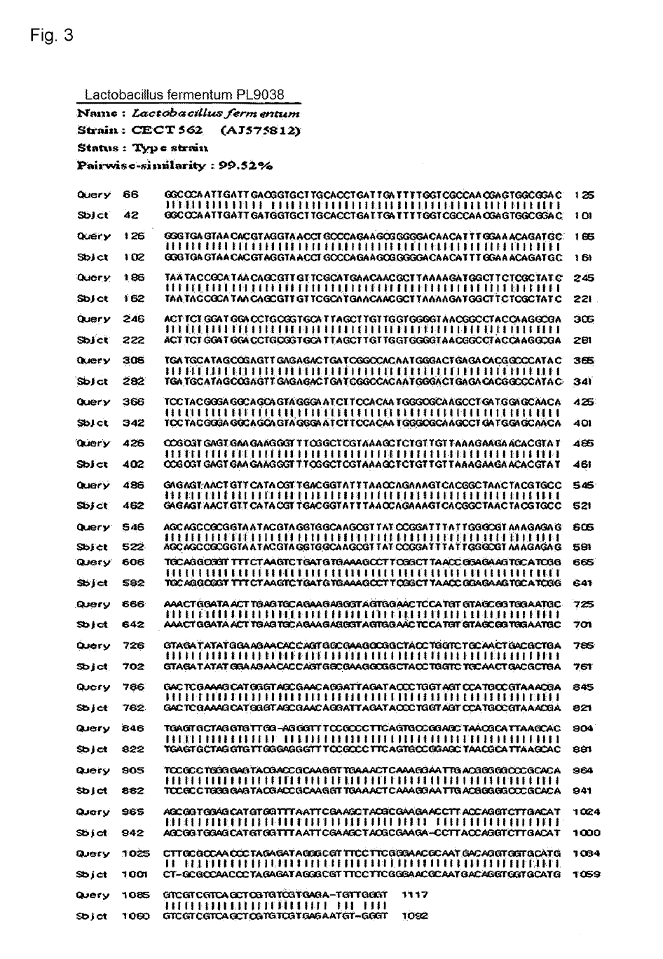

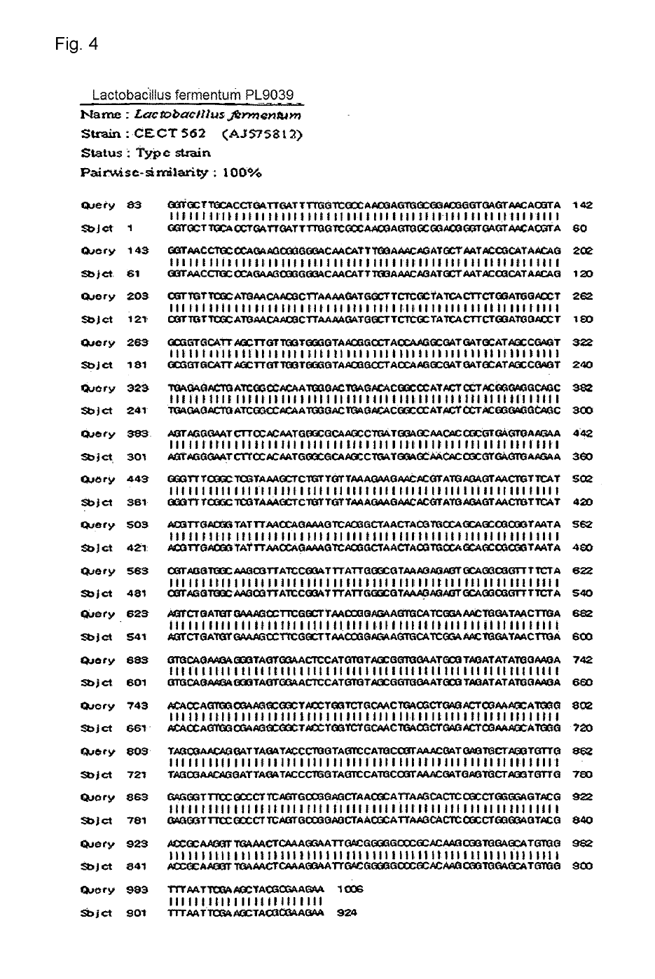

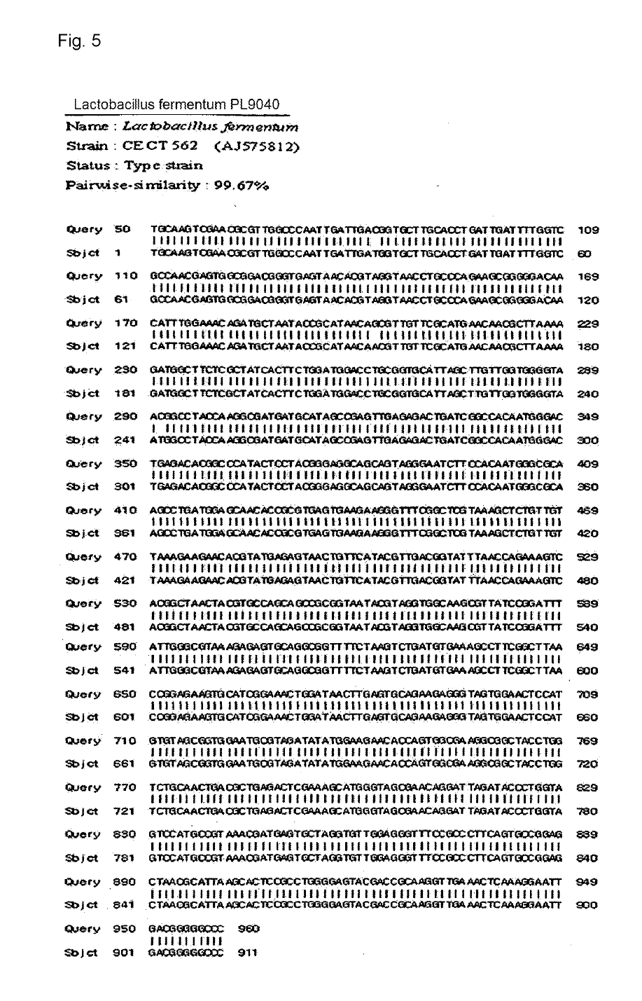

The inventors of the present invention isolated more strains from the human feces samples and identified them. Four kinds of Lactobacillus fermentum strains were confirmed, which showed homologies of over 99% as compared to the standard strain CECT562 of the Lactobacillus fermentum through the 16S rRNA base sequence. The above strains were named the Lactobacillus fermentum PL9037, PL9038, PL9039 and PL9040 (refer to FIGS. 2 to 5).

The present invention provides a probiotic composition which includes as an active ingredient a novel Lactobacillus fermentum PL9988 deposited with a deposit number of KCTC12624BP, a novel Lactobacillus fermentum PL9037, a novel Lactobacillus fermentum PL9038, a novel Lactobacillus fermentum PL9039, a novel Lactobacillus fermentum PL9040, or a culture medium thereof.

The novel Lactobacillus fermentum PL9988 strain has a 16S rRNA base sequence indicated with a sequence number 1, and the novel Lactobacillus fermentum PL9037 strain has a 16S rRNA base sequence indicated with a sequence number 2, the novel Lactobacillus fermentum PL9038 strain has a 16S rRNA base sequence indicated with a sequence number 3, the novel Lactobacillus fermentum PL9039 strain has a 16S rRNA base sequence indicated with a sequence number 4, and the novel Lactobacillus fermentum PL9040 strain has a 16S rRNA base sequence indicated with a sequence number 5.

The culture medium of the above microorganisms may contain various antibacterial organic acids and non-protein antibacterial components.

Other kinds of known microorganisms which is good to eat together with the lactic acid bacteria of the present invention and inhibit the growth of harmful microorganism and provide activations to improve the balance of intestinal colony can be added to the composition of the present invention.

The above strain does not have any internal and external resistances (transfer possibility) with respect to the antibiotics, for example, gentamicin, kanamycin, streptomycin, neomycin, tetracycline, erythromycin, clindamycin, chloramphenicol, ampicillin, synercid (quinupristin and dalfopristin combination), linezolid, trimethoprim, ciprofloxacin and rifampicin, and has an internal resistance with respect to vancomycin which does not have any resistance transfer possibility.

The strain has a good intestine cell adhesiveness, an acid resistance and a bile resistance and may provide an antibacterial effect, an anti-oxidation effect, an immunity enhancement activation and an anti-toxicity shock inhibition effect.

According to a detailed exemplary embodiment of the present invention, in order to confirm an antibiotic sensitivity of the Lactobacillus fermentum strain, the inventors of the present invention subjected, in accordance with an IOS guideline, gentamicin, tetracycline, erythromycin, clindamycin, chloramphenicol, ampicillin, synercid, linezolid and rifampicin, which are antibiotics, to the Lactobacillus fermentum PL9988 strain and the Lactobacillus fermentum PL9037, PL9038, PL9039 and PL9040 strains, respectively, and as a result of the liquid dilution process, it was confirmed that as compared to other Lactobacillus fermentum strains, the Lactobacillus fermentum PL9988 strain had a sensitivity with respect to all the antibiotics, so it did not have any problem with the antibiotic resistance transfer (refer to Table 1).

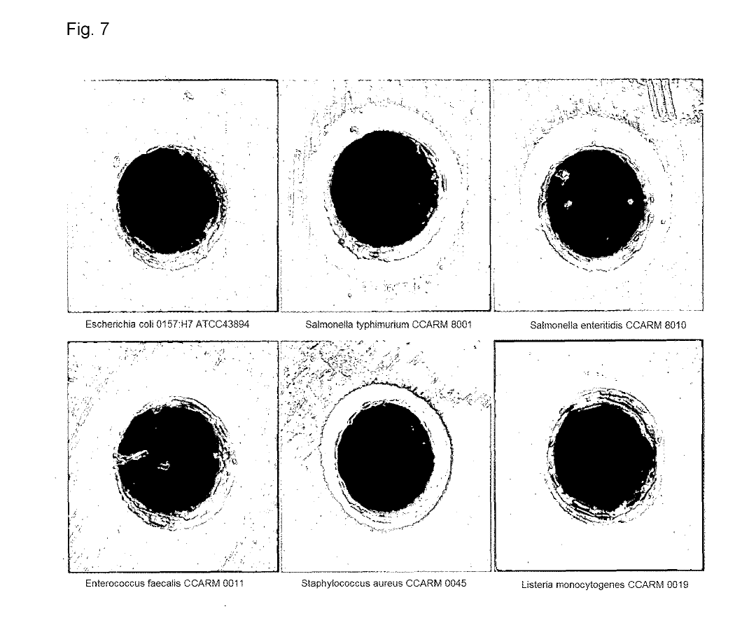

In order to confirm any pathogenic inhibition capability of the Lactobacillus fermentum strains, the inventors of the present invention cultured six harmful bacteria consisting of Escherichia coli 0157:H7 ATCC43894, Salmonella typhimurium CCARM 8001, Salmonella enteritidis CCARM 8010, Enterococcus faecalis CCARM 0011, Staphylococcus aureus CCARM 0045, Listeria monocytogenes CCARM 0019 and Lactobacillus fermentum strains and confirmed the presence and size of the inhibition zone. As a result, the Lactobacillus fermentum PL9988 strain, and the Lactobacillus fermentum PL9037, PL9038, PL9039 and PL9040 strains showed meaningful pathogenic bacteria inhibitions. In particular, the Lactobacillus fermentum PL9988 strain killed five kinds of harmful bacteria except for the Escherichia coli 0157:H7, which meant that it has a good pathogenic bacterium inhibition (refer to FIG. 7 and Table 2).

In order to confirm the stability of the Lactobacillus fermentum strains, the inventors of the present invention conducted a hemolysis phenomenon inspection, and production inspections on a harmful substance, for example, urea, indole, phenylpyruvic acid, etc. and of a harmful enzyme, for example, β-glucuronidase, β-gluscosidase, etc. and a gelatin liquefaction reaction inspection. As a result, as compared to the Lactobacillus fermentum PL9037, PL9038, PL9039 and PL9040 strains, the Lactobacillus fermentum PL9988 strain did not show any hemolysis phenomenon and did not product any harmful substance and harmful enzyme and showed a negative result in terms of the liquefaction reaction, which meant that it had the best stability (refer to Table 3).

In order to confirm the acid resistance and bile resistance of the Lactobacillus fermentum strain, the inventors of the present invention added an artificial gastric juice and bile to the strain, and the number of the living bacteria was measured after cultivation. As a result, it was confirmed that since most of the strains survived, the Lactobacillus fermentum PL9988 strain had a good acid resistance and a good bile resistance (refer to Table 4).

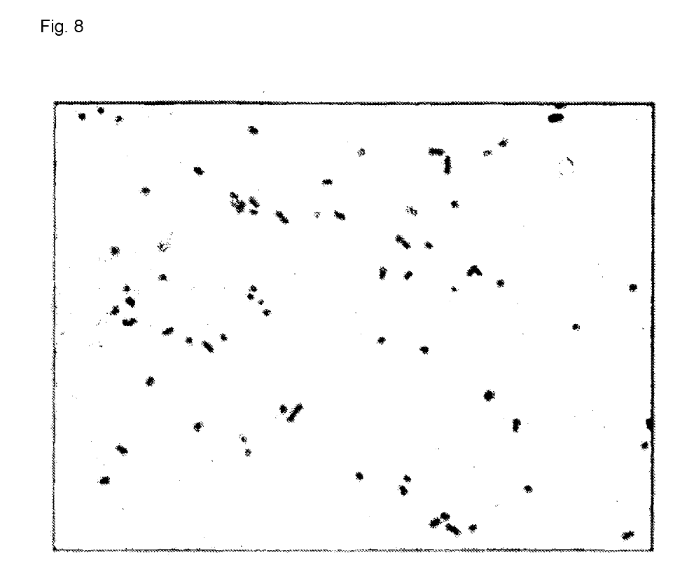

In order to confirm an adhesion to the intestinal cell of the Lactobacillus fermentum PL9988 strain, the inventors of the present invention subjected the lactic acid bacteria to the human intestine cell line, and the number of adhered living bacteria was measured using the Gram staining and continuous dilution method. As a result, it was confirmed that the adhesion of the Lactobacillus fermentum PL9988 strain was good (refer to FIG. 8).

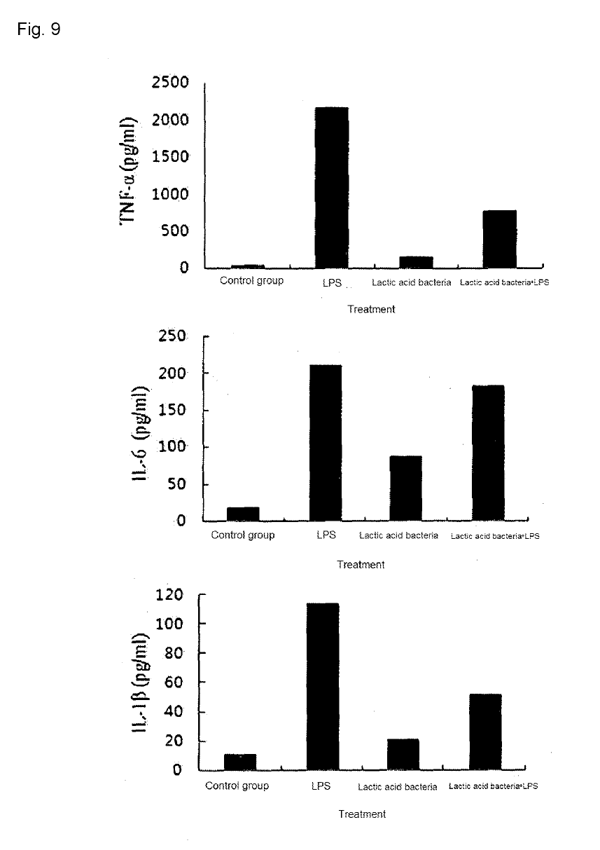

In order to confirm the immunity enhancement effect of the Lactobacillus fermentum PL9988 strain, the inventors of the present invention subjected the Lipopolysaccharide (LPS) and the Lactobacillus fermentum PL9988 strain to macrophage cell lines and measured the concentration of TNF-α, IL-6 and IL-1β which are proinflammatory cytokine. As a result, in case where the Lactobacillus fermentum PL9988 strain was subjected, it was confirmed that the concentrations of three occasions were increased, and an immunity enhancement effect was obtained. In addition, in case where the LPS was singularly subjected, the concentrations of TNF-α, IL-6 and IL-1β were increased, and in case where the LPS and the Lactobacillus fermentum PL9988 strain were simultaneously subjected, it was confirmed that the concentrations of TNF-α, IL-6 and IL-1β were decreased. To this, it was confirmed that the Lactobacillus fermentum PL9988 strain had an effect on minimizing the anti-toxicity shock (refer to FIG. 9).

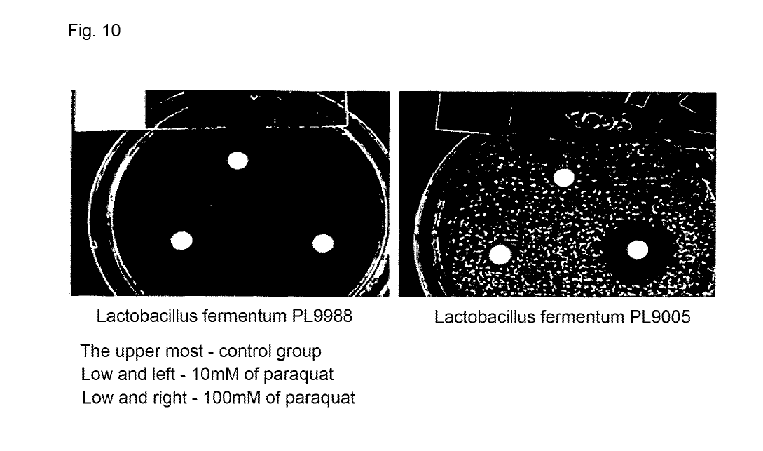

In order to confirm any anti-oxidation effect of the Lactobacillus fermentum PL9988 strain, the inventors of the present invention conducted an anti-oxidation function measurement test. As a result, it was confirmed that the Lactobacillus fermentum PL9988 strain had a resistance with respect to paraquat since any growth inhibition zone was not observed in both cases when 10 mM and 100 mM of paraquat, which generates superoxide anion, were added. For this reason, it was confirmed that the Lactobacillus fermentum PL9988 strain had a good anti-oxidation capability (refer to FIG. 10 and Table 5).

So, the novel Lactobacillus fermentum strain and the culture medium thereof according to the present invention have a good intestinal cell adhesiveness and a good acid resistance and a good bile resistance and don't have any problem with the antibiotic resistance transfer and are able to provide an intestinal harmful pathogenic bacteria inhibition, an immunity enhancement effect, an anti-toxicity shock inhibition effect and an anti-oxidation effect. In particular, since the Lactobacillus fermentum PL9988 strain meaningfully satisfies the conditions as probiotic, the novel Lactobacillus fermentum PL9988 and the culture medium thereof may be used as a probiotic composition which may enhance defecation and intestinal health.

The composition according to the present invention may be prepared based on a typical probiotic composition preparation method and may in general be a cultivation suspension or a dried powder. The composition according to the present invention may be prepared in a composition form of a typical formulation in such a way that one or more than two kinds of pharmaceutically acceptable typical carriers or one or more than two kinds of additives are selected and added to the effective does of the Lactobacillus fermentum PL9988 strain or the culture medium thereof which is the main ingredient.

The carrier may be prepared in such a way to select one or more than two kinds among a diluent, a modifier, a binder, a disintegrating agent, a sweetener, a stabilizer, and an antiseptic. The additive may be prepared in such a way to select one or more than two kinds among a perfume, vitamins and an anti-oxidizing agent.

In the present invention, the carrier and additives may be all kinds of pharmaceutically acceptable ones. More specifically, the diluent may be lactose monohydrate, Trehalose, cornstarch, soybean oil, microcrystalline celluose or D-mannitol, and the modifier may preferably be magnesium stearate or talc, and the binder may be preferably selected from the group consisting of polyvinyipyrolidone (PVP) and hydroxypropylcellulose (HPC). In addition, the disintegrating agent may be preferably selected from the group consisting of carboxymethylcellulose calcium (Ca-CMC), sodium starchglycolate, polacrylin potassium, and cross-linked polyvinylpyrrolidone, and the sweetener may be selected from the group consisting of sucrose, fructose, sorbitol and aspartame, and the stabilizer may be selected from the group consisting of carboxymethylcellulose sodium, β-cyclodextrin, white bee's wax and xanthan, and the antiseptic may be selected from the group consisting of methyl p-hydroxybenzoate methlparaben, propyl p-hydroxybenzoate propylparaben and potassium sorbate.

The present invention provides a health food which includes a novel Lactobacillus fermentum strain PL9988 deposited with a deposit number of KCTC12624BP, a novel Lactobacillus fermentum strain PL9037, a novel Lactobacillus fermentum strain PL9038, a novel Lactobacillus fermentum strain PL9039, a novel Lactobacillus fermentum strain PL9040 or a culture medium thereof as an active ingredient.

The Lactobacillus fermentum PL9988 strain has a 16S rRNA base sequence marked as a sequence number 1, and the novel Lactobacillus fermentum PL9037 strain has a 16S rRNA base sequence marked as a sequence number 2, and the novel Lactobacillus fermentum PL9038 strain has a 16S rRNA base sequence marked as a sequence number 3, and the novel Lactobacillus fermentum PL9039 strain has a 16S rRNA base sequence marked as a sequence number 4, and the novel Lactobacillus fermentum PL9040 strain has a 16S rRNA base sequence marked as a sequence number 5.

The culture medium of the above microorganism may contain various antibacterial organic acids and non-protein antibacterial components.

The composition according to the present invention may include other the lactic acid bacteria which is good to eat, able to inhibit the growth of the harmful microorganism and improve the balance of the intestinal colony. The strains according to the present invention may have a good intestine cell adhesiveness and a good acid resistance and a good bile resistance and may be able to enhance an intestinal health activation through an enhanced antibacterial function and anti-oxidation effect, an immunity enhancement activation, an anti-toxicity shock inhibition and an intestinal pathogenic microorganism growth inhibition.

It is preferred that the above mentioned food may be selected from the group consisting of dairy products formed of ice creams, milk, soybean milk, yogurt, and cheese, meats, sausage, bread, chocolate, candies, snacks, confectionery, pizza, Ramen, other noodles, chewing gums, various soups, beverages, teas, drinks, alcohol drinks, etc.

To this end, the Lactobacillus fermentum strain according to the present invention may be usefully used as a health food which is able to enhance defecation and intestinal health since it has a good intestine cell adhesiveness and a good acid resistance and a good bile resistance and does not have a problem with an antibiotic resistance transfer and is able to inhibit the growths of pathogenic bacteria which are harmful to intestines while obtaining an immunity enhancement effect, an anti-toxicity shock inhibition and an anti-oxidation effect.

As an example of the health food according to the present invention, there is a home remedy agent made to improve intestinal functions, for example, tea, jelly, juice, extract, drinks, etc. which contain, as an active ingredient, the Lactobacillus genus strain or the culture medium thereof. The disease prevention health food according to the present invention which may be processed into various type is easy to take without any side effect to a human body, and it may be stored for a long time.

If the Lactobacillus genus strain or the culture medium thereof is used as a food additive, the above strain or the culture medium thereof may be directly used or may be used together with other foods or food ingredients and may be appropriately used based on a traditional way. The mixing amount of the active ingredients may be appropriately determined based on the use purpose (a prevention, health or remedy treatment). In general, when the food or drinks is prepared, the amount thereof is added by less than 15 parts by weight with respect to 100 parts by weight of the Lactobacillus genus strain or the culture medium thereof, preferably, by less than 10 parts by weight. In case of a long term administration for the sake of health and sanitation or health control, the above amount may be reduced below the above range. Since there is not any problem with stability, the active ingredients may be used higher than the above amount range.

There is no limit to the kinds of foods. As an example of such a food to which the Lactobacillus genus strain or the culture medium thereof may be added, there may be meats, sausage, bread, chocolate, candies, snacks, confectioneries, pizza, Ramen, other noodles, chewing gums, ice confectionery, ice creams, milk, substitutes of milk, cream, butter, butter milk, yogurt, dairy products including cheese, various soups, beverages, tea, drinks, alcohol drinks, vitamin complex, etc. The above food may include all kinds of typically defined health foods.

The health drink composition according to the present invention may contain various flavoring agents or natural carbohydrate, etc. like typical beverages as additional components. The above natural carbohydrate may be a sugar alcohol, for example, monosaccharide, for example, glucose, fructose, etc., disaccharide, for example, maltose, sucrose, etc., polysaccharide, for example, dextrin, cyclodextrin, etc., xylitol, sorbitol, erythritol, etc. The sweetener may be a natural sweetener, for example, thaumatin, stevia extract, etc. or a synthetic sweetener, for example, saccharin, aspartame, etc. The ratio of the natural carbohydrate may be 0.01 to 0.04 g with respect to 100 ml of the Lactobacillus genus strain or the culture medium thereof, and the ratio thereof is preferably about 0.02 to 0.03 g.

The Lactobacillus genus strain or the culture medium according to the present invention may include various nutrients, vitamins, electrolytes, flavoring agents, coloring agents, pectic acids and its bases, alginic acids and its bases, organic acids, protective colloid thickeners, pH modifiers, stabilization agents, antiseptics, glycerin, alcohols, carbonation agents used for carbonated soft drinks, etc. The probiotics according to the present invention may include natural fruit juices, fruit juice beverages and fruit fresh used to prepare vegetable drinks. These components may be independently used or may be used combined. The adding ratio of such additives does not matter, but it is preferred that the strain and its culture medium according to the present invention is in a range of 0.01 to 0.1 parts by weight with respect to 100 parts by weight.

In addition, for the use in a form of a probiotic composition, the present invention may provide a novel Lactobacillus fermentum PL9988 deposited with the deposition number KCTC12624BP, a novel Lactobacillus fermentum PL9037, a novel Lactobacillus fermentum PL9038, a novel Lactobacillus fermentum PL9039, a novel Lactobacillus fermentum PL9040, and the use of their culture medium.

The Lactobacillus fermentum PL9988 strain has a 16S rRNA base sequence marked with a sequence number 1, and the novel Lactobacillus fermentum PL9037 strain has a 16S rRNA base sequence marked with a sequence number 2, and the novel Lactobacillus fermentum PL9038 strain has a 16S rRNA base sequence marked with a sequence number 3, and the novel Lactobacillus fermentum PL9039 strain has a 16S rRNA base sequence marked with a sequence number 4, and the novel Lactobacillus fermentum PL9040 strain has a 16S rRNA base sequence marked with a sequence number 5.

The culture medium of the microorganism may contain various antibacterial organic acids and non-protein antibacterial components.

The composition according to the present invention is good to eat together with the lactic acid bacteria of the present invention. The growth of harmful microorganism may be inhibited by eating them. Other kinds of known microorganisms may be further added, which provide activations to improve the balance of intestinal colony.

The above strain does not have any extrinsic resistances (transfer possibility) with respect to the antibiotics, for example, gentamicin, kanamycin, streptomycin, neomycin, tetracycline, erythromycin, clindamycin, chloramphenicol, ampicillin, synercid (quinupristin and dalfopristin combination), linezolid, trimethoprim, ciprofloxacin and rifampicin. and has an intrinsic resistance with respect to vancomycin which does not have any resistance transfer possibility.

The strain has a good intestine cell adhesiveness, a good acid resistance and a good bile resistance and may provide an antibacterial effect, an anti-oxidation effect, an immunity enhancement activation and an anti-toxicity shock inhibition effect.

To this end, Lactobacillus fermentum strains and the culture medium thereof according to the present invention have a good intestinal cell adhesiveness, a good acid resistance and a good bile resistance, and there is not any danger to the antibiotic resistance transfer, inhibition activity on intestinal harmful pathogenic bacteria an immunity enhancement effect, an anti-toxicity shock inhibition effect, and an anti-oxidation effect. In particular, since the Lactobacillus fermentum PL9988 strain meaningfully satisfies all the above characteristics as probiotics, the Lactobacillus fermentum PL9988 or its culture medium may be used as a probiotic composition which is able to enhance defecation and intestinal health.

Meanwhile, for the use as a health good, the present invention may provide a novel Lactobacillus fermentum PL9988 deposited with a deposit number of KCTC12624BP, a novel Lactobacillus fermentum PL9037, a novel Lactobacillus fermentum PL9038, a novel Lactobacillus fermentum PL9039, a novel Lactobacillus fermentum PL9040, and the use of their culture medium.

The Lactobacillus fermentum PL9988 strain has a 16S rRNA base sequence marked with a sequence number 1, and the novel Lactobacillus fermentum PL9037 strain has a 16S rRNA base sequence marked with a sequence number 2, and the novel Lactobacillus fermentum PL9038 strain has a 16S rRNA base sequence marked with a sequence number 3, and the novel Lactobacillus fermentum PL9039 strain has a 16S rRNA base sequence marked with a sequence number 4, and the novel Lactobacillus fermentum PL9040 strain has a 16S rRNA base sequence marked with a sequence number 5.

The culture medium of the microorganism may contain various antibacterial organic acids and non-protein antibacterial components.

The composition according to the present invention is good to eat together with the lactic acid bacteria of the present invention. The growth of harmful microorganism may be inhibited by eating them. Other kinds of known microorganisms may be further added, which provide activations to improve the balance of intestinal colony.

The strain has a good intestinal cell adhesiveness, a good acid resistance and a good bile resistance and is able to enhance an intestinal health activation with the aid of an antibacterial capability, an anti-oxidation effect, an immunity enhancement activation, an anti-toxicity shock inhibition and an intestinal pathogenic microorganism growth inhibition.

It is preferred that the above food may be selected from the group consisting of dairy products formed of ice creams, milk, soybean milk, yogurt, and cheese, meats, sausage, bread, chocolate, candies, snacks, confectionery, pizza, Ramen, other noodles, chewing gums, various soups, beverages, teas, drinks, alcohol drinks, etc.

To this end, the Lactobacillus fermentum strain according to the present invention has a good intestine cell adhesiveness and a good acid resistance and a good bile resistance and don't have any danger in terms of an antibiotic resistance transfer and are able to inhibit harmful pathogenic bacteria in intestines and have an effect on an immunity enhancement, an anti-toxicity short inhibition, and an anti-oxidation, so it can be used usefully as a health food which is able to enhance defecation and intestinal health.

The present invention will be described in detail along with the exemplary embodiments and the preparing examples.

The exemplary embodiments and preparing examples below are provided only for illustrative purposes, and the contents of the present invention are not limited by such exemplary embodiments and preparing examples.

### Embodiment 1

### Isolations of Strains

The human feces samples were collected from 101 healthy adults who were living at eight villages of Jullabukdo in Korea which were known as a Korean longevity belt and who had regular bowl movement and were delivered in frozen states to the laboratory within six hours. The delivered feces samples were diluted with sterile saline solution and inoculated on the De Man Rogosa (MRS. Difco, Becton Dickinson, Sparks, Md. USA) culture medium and smeared and cultured in an incubator for 48 hours at 37° C. The colonies which looked like lactic acid bacteria were collected, and a single colony was isolated (Lee, H M, Lee Y, 2008, Letters in Applied Microbiology 46, 676-681). In case where the same type of colonies were observed in the same person, the strain was isolated and analyzed up to two colonies at maximum. The strain which showed a negative reaction was selected from the isolated colonies through a catalase reaction. Thereafter the strains were subjected to Gram staining, and a strain which was a Gram positive and a rod form was isolated.

### Embodiment 2

### Identification of Strains

The 16s rRNA gene base sequence was carried out so as to identify the strains isolated in the embodiment 1.

More specifically, the strain was identified using the EzTaxon-e server (http://eztaxon-e-ezbiocloud.net/; Kim et al., 2013) as the base sequence and the blast algorithm at the National Center for Biotechnology Information Web server (http://www.ncbi.nlm.nih.gov), and a molecular phylogeny analysis based on the 16S rRNA base sequence.

As a result, as illustrated in FIG. 1, it was confirmed that the Lactobacillus fermentum PL9988 strain according to the present invention was a novel strain which had a 16s rRNA base sequence marked with a sequence number 1 and a 16S rRNA identification of 99.42% as compared to the standard strain CECT562 (Lactobacillus fermentum strain CECT562) of the Lactobacillus fermentum (FIG. 1). Moreover, the above strain was named the Lactobacillus fermentum PL9988 and was deposited on Jul. 16, 2014 with the deposit number KCTC12624BP at the microorganism resource center of Korea Research Institute of Bioscience and Biotechnology (KRIBB).

In addition, more strains were isolated and identified in the same method.

As a result, as illustrated in FIGS. 2 to 5, four kinds of Lactobacillus fermentum strains were isolated and identified, which were different from the Lactobacillus fermentum PL9988 of the present invention and had the 16S rRNA base sequences marked with sequence numbers 2 to 5 and had the 16S rRNA homologies of over 99% as compared to the Lactobacillus fermentum standard strain CECT562 (FIGS. 2 to 5). The above strains were named the Lactobacillus fermentum PL9037, PL9038, PL9039 and PL9040, respectively.

### Embodiment 3

### Staining of Lactobacillus fermentum PL9988

The Gram staining method was carried out in order to confirm the shape of the Lactobacillus fermentum PL9988 isolated according to the embodiment 1.

More specifically, the above strain was cultured at a nutrition agar flat culture medium at 30° C. for 24 hours so as to analyze the type of the Lactobacillus fermentum PL9988 strain isolated and identified in the embodiments 1 and 2, and the strain was smeared on a glass slide, and the type of the strain and the type of the colony were observed using a microscope, and the Gram staining experiment was carried out using a Gram staining kit (Sigma Diagnostics, Kit HT 90-A) manufactured by Sigma corporation.

As a result, as illustrated in FIG. 6, it was confirmed that the shape of the Lactobacillus fermentum PL9988 strain according to the present invention was the same as the shape of the Gram positive rod form strain of the typical Lactobacillus fermentum (FIG. 6).

### Embodiment 4

### Antibiotic Sensitivity Confirmation of Lactobacillus Fermentum Strain

The liquid dilution method was carried in order to measure the sensitivity to the antibiotics of the Lactobacillus fermentum PL9988 strain isolated in the embodiment 2. The inspection of the sensitivity of the antibiotics of the lactic acid bacteria was disclosed in the ISO (International Organization for Standardization), CSLI (Clinical and Laboratory Standards Institute), and EUCAST (European Committee on Antimicrobial Susceptibility Testing), but any resistance criteria with respect to various species of lactic acid bacteria and antibiotics were not established. Considering this, the resistances were relatively determined in such a way to simultaneously carry out the sensitivity inspections of the above isolated Lactobacillus fermentum strains.

More specifically, the sensitivity inspection of the antibiotics of the lactic acid bacteria was carried by the liquid dilution method by using the combined culture medium (pH 6.7) of the LSM culture medium (IST broth (Iso-Sensitest, 90%; Oxoid) and the MRS broth (10%) based on the ISO guideline (ISO10932: 2010(E), Milk and milk products-determination of the minimal inhibitory concentration (MIC) of antibiotics applicable to bifidobacteria and non-enterococcal lactic acid bacteria (LAB). The experiment was carried within a concentration range of 0.5 to 256 μg/ml by using the antibiotics of gentamicin, tetracycline, erythromycin, clindamycin, chloramphenicol, ampicillin, vancomycin, a combination of synercid quinupristin and dalfoprstin, linezolid, and rifampicin which were suggested in the ISO guideline. The antibiotics to be tested were prepared two times (2×) concentrate, and the thusly prepared antibiotics were divided by 50 μl for each well of the microplate. The lactic acid bacteria cultured overnight were inoculated at the well filled with the antibiotics, and the microplate at which the strains had been inoculated was cultured for 48 hours at 37° C. under an anaerobic condition, and a result thereof was observed.

As a result, as seen in Table 1, it was confirmed that the novel Lactobacillus fermentum strains according to the present invention had a high resistance of the minimal inhibitory concentration (MIC)=over 128 μg/ml with respect to the vancomycin, so it was consequently confirmed that the Lactobacillus fermentum strains had a phenomenon due to a thick inner wall which was a common feature of the lactic acid bacteria. In addition, unlike Lactobacillus fermentum PL9037, PL9038, PL9039 and PL9040 each having one or more resistance with respect to gentamicin, erythromycin, and clindamycin, it was confirmed that the Lactobacillus fermentum PL9988 had a sensitivity all antibiotics. This showed that Lactobacillus fermentum PL9988 strain did not have any danger in terms of the antibiotic resistance transfer (Table 1).

TABLE 1
A result of the measurements of the minimal inhibitory concentration
(MIC) of the Lactobacillus fermentum strains

| Identification | Minimal Inhibitory Concentration (MIC) | | | | | | | | | |
|---|---|---|---|---|---|---|---|---|---|---|
| number | GEN | TET | ERY | CLI | CHL | AMP | VAN | SYN | LIN | RIF |
| PL9988 | 8 | 8 | 1 | 0.06 | 8 | 0.5 | >128 | 1 | 4 | 0.5 |
| PL9037 | 16 | 8 | 8 | 4 | 4 | 0.5 | >128 | 0.5 | 4 | 0.25 |
| PL9038 | 8 | 4 | 8 | 4 | 8 | 0.5 | >128 | 0.5 | 4 | ≦1.125 |
| PL9039 | 16 | 8 | 8 | 0.25 | 8 | 0.5 | >128 | 0.5 | 8 | ≦0.125 |
| PL9040 | 16 | 4 | 4 | 0.5 | 8 | 1 | >128 | 0.5 | 4 | ≦0.125 |

(GEN: gentamicin, TEF: tetracycline, ERY: erythromycin, CLI: clindamycin, CHL: chloramphenicol, AMP: ampicillin, VAN: vancomycin, SYN: synercid LIN: linezolid, and RIF: rifampicin)

### Embodiment 5

### Confirmation of Pathogenic Bacterium Inhibition Capability of Lactobacillus fermentum Strain

The following experiments were carried out so as to measure the pathogenic bacterium inhibition capability of the novel Lactobacillus fermentum strain isolated in the embodiment 1.

More specifically, the supernatant was prepared in such a way that the lactic acid bacteria were cultured for 25 hours at the MRS liquid culture medium and were subjected to centrifugation. Six pathogenic bacteria consisted of Escherichia coli 0157:H7 ATCC43894, Salmonella typhimurium CCARM 8001, Salmonella enteritidis CCARM 8010, Enterococcus faecalis CCARM 0011, Staphylococcus aureus CCARM 0045, and Listeria monocytogenes CCARM 0019 were inoculated at the Mullter Hinton (MH) solid culture medium after the turbidity was adjusted to the McFaland standard 0.5. A hole was made at the culture medium using a sterilized test tube, and 1 ml of the lactic acid bacteria-cultured supernatant was mixed with 200 ml of the agar (3%), and the mixture was added into the hole. Listeria monocytogenes was cultured at 30° C., and the remaining other pathogenic bacteria were cultured at 37° C., and the presence of the inhibition zone and the size thereof were confirmed.

As a result, as seen in FIG. 7 and Table 2, it was confirmed that the Lactobacillus fermentum PL9988 strains had an inhibition activity with respect to various bacteria since it was confirmed that the Lactobacillus fermentum PL9988 strain showed an inhibition activity with respect to five kinds of pathogenic bacteria except for Escherichia coli 0157:H7. Moreover, Lactobacillus fermentum PL9037 and PL9038 strains showed an inhibition activity with respect to five kinds of bacteria, and it was confirmed that Lactobacillus fermentum PL9039 and PL9040 strains showed a pathogenic bacterium inhibition activity similar to the Lactobacillus fermentum PL9988 strain according to the present invention (FIG. 7 and Table 2).

TABLE 2

| Identification | Harmful bacterium inhibition | | | | | |
|---|---|---|---|---|---|---|
| number | EC | ST | SE | EF | SA | LM |
| PL9988 | — | 21 | 21 | 21 | 19 | 23 |
| PL9037 | 18 | 20 | 22 | 21 | — | 29 |
| PL9038 | 17.5 | 21.5 | 21.5 | 21 | — | 28 |
| PL9039 | 16.5 | 21.5 | 20.5 | 20 | 19 | 27 |
| PL9040 | 16.5 | 19.5 | 20 | 20 | 19.5 | 26 |

EC: Escherichia coli 0157: H7 ATCC43894
ST: Salmonella typhimurium CCARM 8001,
SE: Salmonella enteritidis CCARM 8010,
EF: Enterococcus faecalis CCARM 0011,
SA: Staphylococcus aureus CCARM 0045,
LM: Listeria monocytogenes CCARM 0019

### Embodiment 6

### Antibiotic Sensitivity Confirmation of Lactobacillus fermentum Strain

The hemolysis phenomenon inspection, and harmful substance and harmful enzyme generation inspection were carried out so as to measure the safety of a human body after the novel Lactobacillus fermentum strain isolated in the embodiment 1 was used. The pathogenicity of the bacteria was greatly dependent on cell penetration capability. The cell penetration needed a protein degradation capability. A gelatin liquefaction reaction test was carried out so as to confirm the protein degradation capability.

More specifically, the experiment bacteria were inoculated at the blood agar culture medium and cultured for 24 hours at 37° C., and the hemolysis phenomenon was confirmed. The experiment bacteria was inoculated at the MRS gelatin culture medium containing 0.3 g of beef extract, 0.5 g of peptone, 12 g of gelatin and 100 ml of the MRS culture medium and was cultured for 6 weeks at 35° C. It was cooled for 4 hours at 4° C., and the gelatin liquefaction reaction was observed together with the control group which was not inoculated with bacteria. If the culture medium was not hardened after cooling, it was determined as a positive reaction. In addition, the productions of any harmful substances, for example, urease, indole, phenylpyruvic acid, etc. and any harmful enzymes produced by a part of intestinal microorganisms, for example, β-glucuronidase and β-glucosidase were confirmed.

As a result, as seen in Table 3, it was confirmed that the novel Lactobacillus fermentum PL9988 strain was negative in all the gelatin liquefaction reactions, and any of urease, indole, phenylpyruvic acid, etc. which were a harmful metabolism by-product and any of β-glucuronidase and β-glucosidase which were harmful enzymes were not produced.

In contrast, different from Lactobacillus fermentum PL9988 strain, Lactobacillus fermentum PL9039 strain showed a α-type hemolysis phenomenon, and it was confirmed that Lactobacillus fermentum PL9037 strain, Lactobacillus fermentum PL9038 strain and Lactobacillus fermentum PL9040 strain produced harmful metabolism by-products or harmful enzymes. It was confirmed that Lactobacillus fermentum P9988 strain was the safest (Table 3).

TABLE 3

| | | | | | Production of harmful | |
|---|---|---|---|---|---|---|
| | | Production of harmful substances | | | enzymes | |
| Identification | Hemolysis | | | phenylpyruvic | β- | β- |
| number | phenomenon | urease | indole | acid | glucuronidase | glucosidase |
| PL9988 | X | X | X | X | X | X |
| PL9037 | X | ◯ | X | X | X | X |
| PL9038 | X | X | ◯ | X | ◯ | X |
| PL9039 | ◯ | X | X | X | X | X |
| PL9040 | X | X | X | ◯ | X | ◯ |

### Embodiment 7

### Acid Resistance and Bile Resistance of Lactobacillus fermentum Strains

The acid resistance and bile resistance experiments were carried out in order to measure the acid resistance and bile resistance of the Lactobacillus fermentum strain isolated in the embodiment 1.

More specifically, in order to measure under the environment similar to the condition of a digestive duct, the acid resistance experiment was carried out using an artificial gastric juice wherein 1000 U/ml of pepsin was added to the pH 3.0 culture medium. The isolated lactic acid bacterium strain was cultured for 24 hours at the liquid culture medium and was subjected to centrifugation, and the cells were collected and washed three times using a sterilized saline solution. The same amount of artificial gastric juice was added to the the supernatant, and the mixture was reacted for 90 minutes under the same condition as the lactic acid bacterium culture condition. The sample and the control group were diluted and smeared on the MRS plate. The number of living bacteria was counted, and the output of the acid resistance was measured.

Moreover, the bile resistance experiment was carried using the lactic acid bacterium culture medium which was treated for 90 minutes under the artificial gastric juice condition and was carried out using an artificial bile liquid wherein 0.3% of bile (a pig bile extract, Sigma) and 1000 U/ml trypsin was added at the pH 7.0 culture medium. The culture medium processed with artificial gastric juice was subjected to centrifugation, and the artificial bile liquid was added by the same amount as the supernatant, and the mixture was reacted for more 90 minutes, and the reacted mixture was compared to the control group and was diluted and smeared on the MRS plate. The number of living bacteria was counted, and the output of the bile resistance was measured.

As a result, as seen in Table 4, it was confirmed that the Lactobacillus fermentum PL9988 strain, the Lactobacillus fermentum PL9037 strain, the Lactobacillus fermentum PL9038 strain and the Lactobacillus fermentum PL9039 strain had meaningful viabilities even after the processes of the artificial gastric juice and artificial bile liquid. In particular, it was confirmed that most of the Lactobacillus fermentum PL9988 strains survived after the processes of the artificial gastric juice and artificial bile liquid. Tt was possible to confirm that the Lactobacillus fermentum PL9988 strain was excellent at the acid resistance and the bile resistance (Table 4).

TABLE 4

| Identification | MRS | | | Gastric juice | Bile acid |
|---|---|---|---|---|---|
| number | 0 minute | 90 minutes | 180 minutes | 90 minutes | 90 minutes |
| PL9988 | 3.70 × 10⁸ | 4.30 × 10⁹ | 4.70 × 10⁹ | 9.60 × 10⁸ | 4.80 × 10⁸ |
| PL9039 | 3.50 × 10⁸ | 4.10 × 10⁹ | 4.40 × 10⁹ | 7.20 × 10⁸ | 3.10 × 10⁸ |
| PL9038 | 3.60 × 10⁸ | 4.20 × 10⁹ | 4.70 × 10⁹ | 6.80 × 10⁸ | 3.70 × 10⁸ |
| PL9039 | 3.70 × 10⁸ | 4.30 × 10⁹ | 4.60 × 10⁹ | 8.40 × 10⁸ | 3.60 × 10⁸ |
| PL9040 | 3.60 × 10⁸ | 3.90 × 10⁹ | 4.30 × 10⁹ | 6.50 × 10⁸ | 3.80 × 10⁸ |

### Embodiment 8

### Confirmation of Adhesion Capability to Intestinal Cells of Lactobacillus fermentum PL9988 Strain

The following experiments were carried out in order to measure an adhesion capability to human intestinal cells of Lactobacillus fermentum strain isolated in Embodiment 1.

More specifically, the Caco-2 cell (Korea cell line bank, Korea) which is a human intestinal cell line was cultured at a culture device at 37° C. and 7% CO₂ by using the MEM (Eagles, Gibco) culture medium containing 20% inactivated fetal bovine serum (a fetal bovine serum, FBS, Gibco) and 1% (v/v) antifungal drug (antibiotic-antimycotics, Gibco). The Caco-2 cell which grew more than 70% of the plate and the lactic acid bacteria (1×10⁸ CFU/ml) cultured at the MRS broth were washed three times using 10 mM PBS (pH 7.0), and were suspended at 1 ml of the MEM culture medium and were added to the prepared plates, respectively. The lactic acid bacteria were reacted for one hour at a CO₂ culture device at 37° C., and the plates were washed three times using 10 mM PBS, thus eliminating the lactic acid bacteria which had not been adhered, and were fixed using a 4% fixing agent which contained 100 ml of 35% formaldehyde, 16 g of Na₂HPO₄, and 4 g of NaH₂PO₄—H₂O in 1 liter of distilled water and were Gram-stained as described in the embodiment 3. Thereafter, the adhered lactic acid bacteria were observed using a microscope. In addition, the thusly reacted plate was washed three times using 10 mM PBS, and 1 ml of 0.1% TritonX-100 was added, and the cells and lactic acid bacterium suspension was obtained using a scrapper. The number of the lactic acid bacteria grown on the MRS broth plate using the serial dilution method was counted, and the number of the lactic acid bacteria adhered to the human intestinal cells was confirmed. The above adhering experiment was carried out three times. An average value was obtained.

As a result, as seen in FIG. 8, since it was confirmed that about 2.8×10⁶ CFU of Lactobacillus fermentum PL9988 were adhered to the intestinal cells in each field, it was possible to confirm that the Lactobacillus fermentum PL9988 could inhabit at human intestines and could grow under the intestinal condition (FIG. 8).

### Embodiment 9

### Confirmation of Immunity Enhancement Effect of Lactobacillus fermentum PL9988 Strain

If the non-pathogenic bacteria (lactic acid bacteria) stimulate macrophage cells, TNF-α which is a proinflammatory cytokine will be secreted. For this reason, the level of the cytokine mRNA increases, and the IL-10 secretes, so the immunity enhancement effect can be obtained. Considering this operation, the following experiments were carried out in order to confirm the concentrations of TNF-α, IL-6 and IL-1β which secreted from the macrophage by the Lactobacillus fermentum PL9988 strain isolated in the embodiment 1.

More specifically, the macrophage cell line RAW 264.7 (Korean cell line bank, Korea) was cultured at the RPMI 1640 (Roswell Park Memorial Institute-1640, Gibco BRL, Grand island, USA) containing 10% feral bovine serum (Gibco) and 1% antibiotics-antifungal drug (Gibco) and was inoculated at the 96-well plate at 1.0×10⁵/well, and the cells were stimulated using 1 μg/ml of lipopolysaccaride, LPS. Thereafter, each lactic acid bacterium sub-cultured at the MRS broth was washed three times using the PBS, and the washed lactic acid bacteria were suspended at the RPMI 1640 making the number of the final bacteria to be 1.0×10⁸ CFU/well and were divided onto the plates and were cultured at the CO₂ culture device for 24 hours at 37° C. The culture supernatant wherein only the LPS was reacted, the culture supernatant wherein only the lactic acid bacteria were reacted, and the culture supernatant wherein both the LPS and the lactic acid bacteria were reacted were obtained, and the concentrations of the TNF-α, IL-6 and IL-β which were cytokine related with the immunity enhancement residing in the thusly obtained cell culture supernatants were analyzed based on the procedures from the manufacturers using the TNF-αCytokine kit (Biosource, CA, USA). The above experiments were carried out three times, and an average value was obtained.

As a result, as illustrated in FIG. 9, in case where the macrophage cell lines were stimulated by the LPS and the Lactobacillus fermentum PL9988 was treated, it was confirmed that the secretions of the TNF-α, IL-6 and IL-β were meaningfully reduced as compared to the LPS single process group. For this reason, it was possible to confirm that the Lactobacillus fermentum PL9988 strain had an immunity enhancement effect, and in particular, since the over secretions of TNF-α, IL-6 and IL-β could be reduced with the aid of the LPS, it was confirmed that Lactobacillus fermentum PL9988 strain had a function to minimize any anti-toxicity shock which could be induced by the LPS when infected with bacteria (FIG. 9).

### Embodiment 10

### Anti-Oxidation Effect Confirmation of Lactobacillus fermentum PL9988 Strain

The anti-oxidation function measurement experiment was carried out in order to confirm the anti-oxidation effect of the Lactobacillus fermentum PL9988 strain isolated in the embodiment 1.

More specifically, the strain cultured overnight was added to a saline solution, so the suspension of 10⁷ CFU/ml was made. 0.1 ml of the suspension was inoculated at the MRS solid culture medium, and was cultured overnight at 37° C. in a state where a paper disk containing 10 μl of the paraquat melted in the saline solution was placed, and the inhibition zone of the growth was measured using a ruler. Moreover, 10 and 100 mM of paraquat was added to the suspension and was cultured overnight, and the growths of the bacteria were measured using a 600 nm absorbance at every hour, and the resistance capacity of the reactive oxygen species (ROS) was measured. The Lactobacillus fermentum PL9005 marked with a deposit number of KCCM-10250 was used as a control group.

As a result, as seen in FIG. 10 and Table 5, in case of the Lactobacillus fermentum PL9005 strain marked with a deposit number of KCCM-10250, the growth inhibition zone was observed when 100 mM paraquat was added. As for the Lactobacillus fermentum PL9988 strain of the present invention, such a growth inhibition zone was not observed when 10 nM and 100 nM of the paraquat were added, whereupon it was confirmed that there was a resistance with respect to the paraquat. It was confirmed that the Lactobacillus fermentum PL9988 strain had a good anti-oxidation capability (FIG. 10 and Table 5).

TABLE 5
Growth inhibition zone size due to superoxide
anion that paraquat produces

Lactobacillus fermentum

| Paraquat concentration (mM) | PL9988 | PL9005 |
|---|---|---|
| 0 | 0 | 0 |
| 10 | 0 | 0 |
| 100 | 0 | 16 |

Unit (diameter): mm

### Preparation Example 1

### Preparation of Foods

The foods containing Lactobacillus fermentum PL9988 strain and its culture medium according to the present invention were prepared in the following ways.

1-1. Preparation of Cooking Seasoning

Cooking seasoning for intestinal function improvement was prepared in such a way to mix the Lactobacillus fermentum PL9988 strain and its culture medium at 1 to 12 parts by weight with respect to 100 parts by weight of the cooking seasoning.

1-2. Preparation of Soup and Gravies

The soups and gravies for intestinal function improvement were prepared in such a way to add 1 to 12 parts by weight of the Lactobacillus fermentum PL9988 strain and its culture medium to 100 parts by weight of the soups and gravies and meat processed products and noodles.

1-3. Preparation of Dairy Products

1 to 12 parts by weight of the Lactobacillus fermentum P9988 strain and its culture medium of the present invention were added to 100 parts by weight of milk, so various dairy products like butter and ice cream were prepared using the above milk.

1-4. Preparation of Dry Cereal

Dry powder was prepared in such a way that the Lactobacillus fermentum PL9988 strain and its culture medium according to the present invention was subjected to reduced pressure and enrichment in a vacuum enrichment device and was dried by a spraying and hot wind drier, and the thusly dried mixture was crushed into 60 meshes in terms of particle sizes.

The dry cereal was prepared in such a way to mix the thusly prepared grains, seeds nuts and the Lactobacillus fermentum PL9988 strain and its culture medium.

### Preparation Example 2

### Preparation of Beverages

2-1. Preparation of Carbonated Soft Drink

5 to 10 parts by weight of sugar, 0.05 to 0.3 parts by weight of citric acid, 0.005 to 0.02 parts by weight of caramel, 0.1 to 1 parts by weight of vitamin C, and an additive of 10 parts by weight of the Lactobacillus fermentum PL9988 strain and its culture medium according to the present invention were mixed, and the above mixture is mixed with PL9039 or 94 parts by weight of a distilled water were mixed into a syrup. The syrup was sterilized for 20 to 180 seconds at 95 to 98° C., and the mixture was mixed with a cooling water at a ratio of 1:4, and 0.5 to 0.82 parts by weight of a carbonated gas was injected, thus preparing a carbonated soft drink.

2-2. Preparation of Functional Soft Drink

10 parts by weight of the Lactobacillus fermentum PL9988 strain and its culture medium was mixed with 0.1 parts by weight of vitamin C, 5.8 parts by weight of fructose, 3.8 parts by weight of white sugar, 0.12 parts by weight of citric acid, 0.03 parts by weight of malic acid, 0.04 parts by weight of sodium citrate, and 0.02 parts by weight of gardenia blue, and the mixture was fully dissolved in metered water. The dissolved water was adjusted using a metered water to the total amount thereof to be 100 parts by weight.

2-3. Preparation of Health Beverages

Sub-ingredients, for example, 0.5 parts by weight of high fructose corn syrup, 2 parts by weight of oligosaccharides, 2 parts by weight of sugar, 0.5 parts by weight of table salt and 75 parts by weight of water and 10 parts by weight of the Lactobacillus fermentum PL9988 strain and its culture medium were homogeneously mixed and were instantly sterilized and were packed into a small packing container, for example, a glass bottle, a PET bottle, etc., thus preparing a health beverage.

2-4. Preparation of Vegetable Juices

10 g of a vacuum dry product of the Lactobacillus fermentum PL9988 strain and its culture medium was added to 1 liter of tomato or carrot juice, thus preparing a vegetable juice for enhancing health.

2-5. Preparation of Fruit Juices

10 g of a vacuum dry product of the Lactobacillus fermentum PL9988 strain and its culture medium was added to 1 liter of apple or grape juice, thus preparing a fruit juice for enhancing health.

### Preparation Example 3

### Preparation of Fermented Milk

The raw oil wherein the content of fat free oil solid content was adjusted to 8 to 20 parts by weight using nonfat dry milk was sterilized for 15 seconds at 72 to 75° C. The sterilized raw oil was cooled to a predetermined temperature, and the Lactobacillus fermentum PL9988 strain was inoculated at a concentration of 10⁶ CFU/ml and was cultured until pH became 4 to 5. The culture medium was cooled after the completion of the culture. A syrup was prepared in such a way to melt 0.1 to 50 parts by weight of a fruit juice concentrate, 0.1 to 20 parts by weight of dietary fiber, 0.5 to 30 parts by weight of glucose, 0.1 to 15 parts by weigh of oligosaccharides, 0.001 to 10 parts by weight of calcium, 0.0001 to 5 parts by eight of vitamin, etc. The thusly prepared syrup was sterilized and cooled and was mixed at a predetermined ratio with the culture medium and was agitated and packed into a homogenized container, thus preparing a fermented milk.

### Preparation Example 4

### Preparation of Lactic Acid Bacterium Powder

The Lactobacillus fermentum PL9988 strain was inoculated at the MRS culture medium at a concentration of 10⁶ CFU/ml, and a pH-control fermentation was carried out for 18 to 24 hours at 37° C. Bacterial cells were collected through a centrifugation at 10.000×g at 4° C. after the culture of the Lactobacillus fermentum PL9988 strain was completed. The thusly collected cells were mixed at the same amount with a protective agent wherein 2.5% whey and 5% sucrose were contained in 5% of skim milk, and the mixture was powdered by a freeze drier. The thusly prepared dry powder of the Lactobacillus fermentum PL9988 was diluted with trehalose for the number of the living bacteria to be over 1×10¹¹ CFU/g.

### Preparation Example 5

### Preparation of Lactic Acid Bacterium Formulation

The lactic acid bacterium formulation, for example, a lactic acid bacterium food, a digestive, etc. was prepared using the lactic acid bacterium powder prepared in the preparation example 4. 10 parts by weight of oligosaccharides, 20 parts by weight of anhydrous glucose, 5 parts by weight of granulated fructose, 2 parts by weight of vitamin C, 5 parts by weight of fruit powder flavor, 5 parts by weight of aloe, 15 parts by weight of dietary fiber, and 18 parts by weight of Plantago ovata skin were mixed to 20 parts by weight of dried powder of Lactobacillus fermentum PL9988 strain (the number of living bacteria of over 1×10¹⁰ CFU/g) and divided into a stick container or a bottle by a predetermined amount and was packed. The thusly prepared lactic acid bacterium formulation maintained the number of living bacteria of over 5×10⁸ CFU/g.

### Deposit Number

Name of deposit organ: Microorganism resource center of Korea Research Institute of Bioscience and Biotechnology

Deposit number: KCTC12624BP

Date of deposit: 20140716

1. A novel Lactobacillus fermentum strain.

2. The strain of claim 1, wherein the strain is any of a novel Lactobacillus fermentum PL9988 strain which has a 16S rRNA base sequence marked with a sequence number 1 and were deposited as a deposit number of KCTC12624BP, a novel Lactobacillus fermentum PL9037 strain which has a 16S rRNA base sequence marked with a sequence number 2, a novel Lactobacillus fermentum PL9038 strain which has a 16S rRNA base sequence marked with a sequence number 3, a novel Lactobacillus fermentum PL9039 strain which has a 16S rRNA base sequence marked with a sequence number 4, or a novel Lactobacillus fermentum PL9040 strain which has a 16S rRNA base sequence marked with a sequence number 5.

3. A probiotic composition, comprising:
Any of a novel Lactobacillus fermentum PL9988 deposited as a deposit number of KCTC12624BP; a novel Lactobacillus fermentum PL9037; a novel Lactobacillus fermentum PL9038; a novel Lactobacillus fermentum PL9039; a novel Lactobacillus fermentum PL9040; or a culture medium thereof as active ingredients.

4. The strain of claim 3, wherein the strain or the culture medium thereof does not have any antibiotic extrinsic resistance, so there is no danger to a resistance transfer.

5. The strain of claim 3, wherein the strain or the culture medium thereof has an antibacterial activation.

6. The strain of claim 3, wherein the strain or the culture medium thereof has a good acid resistance and a good bile resistance.

7. The strain of claim 3, wherein the strain or the culture medium thereof has a good intestinal cell adhesiveness.

8. The strain of claim 3, wherein the strain or the culture medium thereof has an anti-oxidation activity.

9. The strain of claim 3, wherein the strain or the culture medium thereof has an immunity enhancement activity.

10. The strain of claim 3, wherein the strain or the culture medium thereof has an immunity enhancement activity in such a way to inhibit the secretion of TNF-α, IL-6 or IL-1β.

11. A health food, comprising:
Any of a novel Lactobacillus fermentum PL9988 deposited as a deposit number of KCTC12624BP; a novel Lactobacillus fermentum PL9037; a novel Lactobacillus fermentum PL9038; a novel Lactobacillus fermentum PL9039; a novel Lactobacillus fermentum PL9040; or a culture medium thereof as active ingredients

12. The health food of claim 11, wherein the strain or the culture medium allows to enhance an intestinal health activity in such a way to inhibit an intestinal pathogenic microorganism growth.

13. The health food of claim 11, wherein the food is one selected from the group consisting of a dairy product formed of ice creams, milk, soybean milk, yogurt, and cheese, a soybean milk product, meats, sausage, bread, chocolate, candies, snacks, confectionery, pizza, Ramen, other noodles, chewing gums, various soups, beverages, teas, drinks, and alcohol drinks.

14. A use of any of a novel Lactobacillus fermentum PL9988 deposited as a deposit number of KCTC12624BP; a novel Lactobacillus fermentum PL9037; a novel Lactobacillus fermentum PL9038; a novel Lactobacillus fermentum PL9039; a novel Lactobacillus fermentum PL9040; or a culture medium thereof as a probiotic composition.

15. A use of any of a novel Lactobacillus fermentum PL9988 deposited as a deposit number of KCTC12624BP; a novel Lactobacillus fermentum PL9037; a novel Lactobacillus fermentum PL9038; a novel Lactobacillus fermentum PL9039; a novel Lactobacillus fermentum PL9040; and a culture medium thereof as a health food.
